(19) United States
(12) Patent Application Publication
(10) Pub. No.: US 2004/0224988 A1
(43) Pub. Date: Nov. 11, 2004

(54) Dosage forms and methods of treatment using VEGFR inhibitors

(75) Inventors: James Freddo, Del Mar, CA (US); Dana Hu-Lowe, Encinitas, CA (US); Yazdi Kersi Pithavala, San Diego, CA (US); Heidi Steinfeldt, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/816,242

(22) Filed: Apr. 1, 2004

Related U.S. Application Data
(60) Provisional application No. 60/460,695, filed on Apr. 3, 2003.
(60) Provisional application No. 60/491,771, filed on Jul. 31, 2003.

(51) Int. Cl. A61K 31/4439; C 07D 4 3/02

(52) U.S. Cl. 514/338; 546/275.7

(57) ABSTRACT

The invention provides dosage forms of a compound of formula 1:

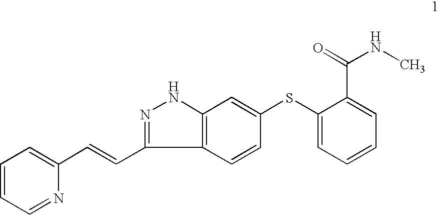

or pharmaceutically acceptable salts, solvates or prodrugs thereof. The invention further provides methods of treating abnormal cell growth, such as cancers, by administering the dosage forms to a mammal.

This application claims the benefit of U.S. Provisional Application No. 60/460,695, filed Apr. 3, 2003, and U.S. Provisional Application No. 60/491,771, filed Jul. 31, 2003, the disclosures of which are incorporated herein by reference in their entireties.

### BACKGROUND OF THE INVENTION

This invention relates to VEGFR inhibitors that are useful in the treatment of abnormal cell growth, such as cancer, in mammals. This invention also relates to a method of using such compounds in the treatment of abnormal cell growth in mammals, especially humans, and to pharmaceutical compositions containing such compounds.

The compound 6-[2-(methylcarbamoyl)phenylsulfanyl]-3-E-[2-(pyridin-2-yl)ethenyl]indazole, represented by formula 1

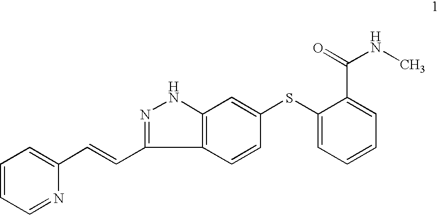

is a potent and selective inhibitor of VEGFR/PDGFR tyrosine kinases with broad preclinical activity in xenograft models of colon, melanoma, breast and lung cancer. (Hu-Lowe D, Heller, D, Brekken J, Feeley R, Amundson K, Haines M, Troche G, Kim Y, Gonzalez D, Herrman M, Batugo M, Vekich S, Kania R, McTigue M, Gregory S, Bender S, Shalinsky D., Pharmacological Activities of AG013736, a Small Molecule Inhibitor of VEGF/PDGF Receptor Tyrosine Kinases; Proc. Am. Assoc. Cancer Res. 2002: abstract #5357). Preclinical tumor vascular response assessed using dynamic contrast enhanced MRI (dceMRI) has been shown to correspond with tumor growth index. (Wilmes L J, Hylton N M, Wang D, Fleming L M Gibbs J, Kim Y, Dillon R, Brasch R C, Park J W, Li K-L, Henry R G, Partridge S C, Shalinsky D R, Hu-Lowe D, McShane TM, and Pallavicini M G., AG013736, a Novel VEGFR TK Inhibitor, Suppresses Tumor Growth and Vascular Permeability in Human BT474 Breast Cancer Xenografts in Nude Mice”; Proc. Am. Assoc. Cancer Res. 2003: Abstract #3772.)

### SUMMARY OF THE INVENTION

The invention provides dosage forms and methods of treatment using a compound of formula 1:

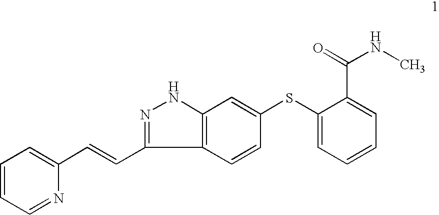

which can be systematically named as 6-[2-(methylcarbamoyl)phenylsulfanyl]-3-E-[2-(pyridin-2-yl)ethenyl]indazole.

In one embodiment, the invention provides a dosage form for administration to a mammal, the dosage form comprising the compound of formula 1, a pharmaceutically acceptable salt, solvate or prodrug thereof, or a mixture thereof, in an amount effective to provide a 24-hour AUC blood plasma value of no more than 4500 ng·hr/mL of the compound of formula 1 or active metabolites thereof, after administration to the mammal. 24-hour AUC blood plasma values can be determined as described in the Detailed Description herein.

In specific aspects of this embodiment, the upper limit of the 24-hour AUC blood plasma value is no more than 4000 ng·hr/mL or no more than 3000 ng·hr/mL or no more than 2500 ng·hr/mL or no more than 2000 ng·hr/mL or no more than 1500 ng·hr/mL or no more than 1000 ng·hr/mL or no more than 800 ng·hr/mL or no more than 700 ng·hr/mL. Preferably, and in combination with any of the recited upper limits, the 24-hour AUC blood plasma value is at least 10 ng·hr/mL or at least 25 ng·hr/mL or at least 50 ng·hr/mL or at least 75 ng·hr/mL or at least 100 ng·hr/mL or at least 125 ng·hr/mL. Contemplated ranges of 24-hour AUC blood plasma values include ranges from any of the recited lower limits to any of the recited upper limits. Specific, non-limiting examples of preferred ranges include from 25 to 4500 ng·hr/mL, 50 to 2500 ng·hr/mL, 75 to 1000 ng·hr/mL, 100 to 800 ng·hr/mL, and 125 to 700 ng·hr/mL.

In another embodiment, the invention provides a dosage form comprising the compound of formula 1 as defined above, a pharmaceutically acceptable salt, solvate or prodrug thereof, or a mixture thereof, in an amount of no more than 30 mg. It should be appreciated that when all or part of the compound is in the dosage form as a salt, solvate or prodrug, the amount is the equivalent amount of the compound of formula 1, which is readily calculated by one skilled in the art based on molar masses.

In specific aspects of this embodiment, the upper limit of the amount is no more than 20 mg or no more than 15 mg or no more than 12 mg or no more than 10 mg or no more than 8 mg or no more than 7 mg. Preferably, and in combination with any of the recited upper limits, the amount is at least 0.5 mg or at least 1 mg or at least 1.5 mg or at least 2 mg or at least 2.5 mg or at least 3 mg. Contemplated ranges include ranges from any of the recited lower limits to any of the recited upper limits. Specific, non-limiting examples of preferred ranges include from 0.5 to 30 mg, 1 to 20 mg, 1.5 to 15 mg, 2 to 10 mg, 2.5 to 8 mg, and 3 to 7 mg.

The invention further provides a method of treating abnormal cell growth in a mammal, including a human, by administering to the mammal the compound of formula 1 as defined above, a pharmaceutically acceptable salt, solvate or prodrug thereof, or a mixture thereof, in an amount effective to provide a 24-hour AUC blood plasma value of no more than 4500 ng·hr/mL of the compound of formula 1 or active metabolites thereof, after administration to the mammal. 24-hour AUC blood plasma values can be determined as described in the Detailed Description herein.

In specific aspects of this embodiment, the upper limit of the 24-hour AUC blood plasma value is no more than 4000 ng·hr/mL or no more than 3000 ng·hr/mL or no more than 2500 ng·hr/mL or no more than 2000 ng·hr/mL or no more than 1500 ng·hr/mL or no more than 1000 ng·hr/mL or no more than 800 ng·hr/mL or no more than 700 ng·hr/mL. Preferably, and in combination with any of the recited upper limits, the 24-hour AUC blood plasma value is at least 10 ng·hr/mL or at least 25 ng·hr/mL or at least 50 ng·hr/mL or at least 75 ng·hr/mL or at least 100 ng·hr/mL or at least 125 ng·hr/mL. Contemplated ranges of 24-hour AUC blood plasma values include ranges from any of the recited lower limits to any of the recited upper limits. Specific, non-limiting examples of preferred ranges include from 25 to 4500 ng·hr/mL, 50 to 2500 ng·hr/mL, 75 to 1000 ng·hr/mL, 100 to 800 ng·hr/mL, and 125 to 700 ng·hr/mL.

The invention further provides a method of treating abnormal cell growth in a mammal, including a human, by administering to the mammal the compound of formula 1 as defined above, a pharmaceutically acceptable salt, solvate or prodrug thereof, or a mixture thereof, in an amount of no more than 30 mg per dose. It should be appreciated that when all or part of the compound is in the dosage form as a salt, solvate or prodrug, the amount is the equivalent amount of the compound of formula 1, which is readily calculated by one skilled in the art based on molar masses.

In specific aspects of this embodiment, the upper limit of the amount is no more than 20 mg or no more than 15 mg or no more than 12 mg or no more than 10 mg or no more than 8 mg or no more than 7 mg. Preferably, and in combination with any of the recited upper limits, the amount is at least 0.5 mg or at least 1 mg or at least 1.5 mg or at least 2 mg or at least 2.5 mg or at least 3 mg. Contemplated ranges include ranges from any of the recited lower limits to any of the recited upper limits. Specific, non-limiting examples of preferred ranges include from 0.5 to 30 mg, 1 to 20 mg, 1.5 to 15 mg, 2 to 10 mg, 2.5 to 8 mg, and 3 to 7 mg.

In a specific embodiment of any of the inventive methods described herein, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In another embodiment, the invention provides a method of inhibiting PDGFR BB mediated cancer cell migration in a mammal, by administering to the mammal a therapeutically acceptable amount of the compound of formula 1.

In another embodiment, the invention provides a method of inhibiting c-KIT activity in a mammal, by administering to the mammal a therapeutically acceptable amount of the compound of formula 1.

In further specific embodiments of any of the inventive methods described herein, the method further comprises administering to the mammal an amount of one or more substances selected from anti-tumor agents, anti-angiogenesis agents, signal transduction inhibitors, and antiproliferative agents, which amounts are together effective in treating said abnormal cell growth. Such substances include those disclosed in PCT publication nos. WO 00/38715, WO 00/38716, WO 00/38717, WO 00/38718, WO 00/38719, WO 00/38730, WO 00/38665, WO 00/37107 and WO 00/38786, the disclosures of which are incorporated herein by reference in their entireties.

Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as paclitaxel (Taxol™), docetaxel (Taxotere™), 2′-N-[3-(dimethylamino)propyl]glutaramate (Taxol™ derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethylnitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2,4-diamino-5-methyl-6-quinazolinyl)methyl]amino]benzoyl]-L-aspartic acid, N-[4-[[(2,4-diamino-5-ethyl-6-quinazolinyl)methyl]amino]benzoyl]-L-aspartic acid, N-[2-chloro-4-[[(2,4-diaminopteridinyl)methyl]amino]benzoyl]-L-aspartic acid, soluble Bakers antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloproteinase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Examples of MMP inhibitors include AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R,3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

3-[4-(4-chloro-phenoxy)-benzenesulfonylamino]-tetrahydro-pyran-3-carboxylic acid hydroxyamide;

(2R,3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined or co-administered with a compound of formula 1. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody bevacizumab (Genentech, Inc. of South San Francisco, Calif.); and angiozyme™, a synthetic ribozyme from Ribozyme (Boulder, Colo.) and Chiron (Emeryville, Calif.).

ErbB2 receptor inhibitors, such as GW-282974 (Glaxo Wellcome pic), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following United States patent applications: Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

The compound of formula 1 may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

In another embodiment, the invention provides a pharmaceutical composition comprising the compound of formula 1, or a pharmaceutically acceptable salt, solvate or prodrug thereof, and a therapeutically effective amount of docetaxel.

In another embodiment, the invention provides a method of treating abnormal cell growth in a mammal, including a human, by administering to the mammal the compound of formula 1, or a pharmaceutically acceptable salt, solvate or prodrug thereof, and a therapeutically effective amount of docetaxel. The compound of formula 1 and docetaxel can be administered separately or in the same composition, and can be administered on the same dosing schedule or on different dosing schedules, as desired.

Definitions

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycolylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

The term “prodrug”, as used herein, unless otherwise indicated, means compounds that are drug precursors, which following administration, release the drug in vivo via some chemical or physiological process (e.g., a prodrug on being brought to the physiological pH is converted to the desired drug form).

The subject invention also includes isotopically-labeled compounds, which are identical to those recited in Formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts and solvates of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula 1 of this invention and prodrugs thereof can generally be prepared by carrying out the procedures described for the non-labeled compound, substituting a readily available isotopically labeled reagent for a non-isotopically labeled reagent.

### BRIEF DESCRIPTION OF THE DRAWINGS

FIG. 1 shows metabolites of the compound of formula 1 identified in dogs following a single oral dose of the ¹⁴C-labeled compound.

FIG. 2 shows metabolites of the compound of formula 1 identified in mice following a single oral dose of the ¹⁴C-labeled compound.

### DETAILED DESCRIPTION OF THE INVENTION

The compound of formula 1 can be prepared as described in U.S. Pat. Nos. 6,531,491 and 6,534,524 (issued Mar. 11, 2003 and Mar. 18, 2003, respectively), which are incorporated herein by reference in their entireties. Certain starting materials may be prepared according to methods familiar to those skilled in the art and certain synthetic modifications may be done according to methods familiar to those skilled in the art.

The compound of formula 1 is capable of forming a wide variety of different salts with various inorganic and organic acids. Although such salts must be pharmaceutically acceptable for administration to mammals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Administration of the compound of formula 1 can be effected by any method that enables delivery of the compound to the site of action. These methods include oral routes, intraduodenal routes, parenteral injection (including intravenous, subcutaneous, intramuscular, intravascular or infusion), topical, and rectal administration.

The compound may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository. The compound may be in unit dosage forms suitable for single administration of precise dosages. Preferably, dosage forms include a conventional pharmaceutical carrier or excipient and the compound of formula 1 as an active ingredient. In addition, dosage forms may include other medicinal or pharmaceutical agents, carriers, adjuvants, etc.

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

In preferred embodiments of the dosage forms of the invention, the dosage form is an oral dosage form, more preferably, a tablet or a capsule.

In preferred embodiments of the methods of the invention, the compound of formula 1 is administered orally, such as, for example, using an oral dosage form as described herein.

The methods include administering the compound of formula 1 using any desire dosage regimen. In one specific embodiment, the compound is administered once per day (quaque die, or QD), preferably twice per day (bis in die, or BID), although more or less frequent administration is within the scope of the invention. The compound can be administered to the mammal, including a human, preferably in a fasted state (no food or beverage within 2 hours before and after administration). In a particularly preferred embodiment, the dosage is BID, fasted.

Methods of preparing various dosage forms with a specific amount of the compound of formula 1 are known, or will be apparent, to those skilled in this art. For examples, see Remington's Pharmaceutical Sciences, Mack Publishing Company, Easter, Pa., 15th Edition (1975).

AUC blood plasma values can be determined by directly measuring blood plasma concentrations of the compound of formula one or active metabolites thereof, such as by liquid chromatography-tandem mass spectrometry (LC-MS/MS), at various time intervals, and calculating the area under the plasma concentration versus time curve. Suitable methods for calculating AUC are well-known in the art, such as, for example, by using the trapezoidal approximation,

$${AUC}_{( 0 - t )} = {\sum }_{i = 0}^{n - 1} \frac{{t}_{i + 1} - {t}_{i}}{2} ( {C}_{i} + {C}_{i + 1} )$$

where n is the number of data points, and tᵢ and Cᵢ are the time and concentration (x and y values) of the ith data point. 24-hour AUC values can be determined by normalizing measured blood plasma concentrations according to the dosing schedule. Sodium bisulfite is added as a stabilizer in the reconstitution solution for preparation of concentration standards.

The compound of formula 1 has advantageous properties relating to the modulation and/or inhibition of the kinase activity associated with VEGF-R, FGF-R, CDK complexes, CHK1, CSF-R, and/or LCK.

As shown in the examples below, the compound of formula 1 is capable of inducing HUVEC apoptosis in vitro, inhibiting VEGF mediated Akt and eNOS phosphorylation in HUVEC, demonstrating a lasting inhibitory effect on VEGFR-2 phosphorylation in HUVEC after compound withdrawal, and inhibiting PDGF BB induced cancer cell migration on matrix protein fibronectin. The compound of formula 1 may have activity against PDGFR-driven tumor progression by inhibiting migration and invasion.

The compound of formula 1 also demonstrates more efficacious activity in tumor growth inhibition when combined with Taxol™, more preferably docetaxel. More significant tumor regression was observed with the co-therapy than either agent alone.

The present invention is further directed to methods of modulating or inhibiting protein kinase activity, for example in mammalian tissue, by administering the compound of formula 1. The activity of the inventive compound as a modulator of protein kinase activity, such as the activity of kinases, may be measured by any of the methods available to those skilled in the art, including in vivo and/or in vitro assays. Examples of suitable assays for activity measurements include those described in Parast C. et al., BioChemistry, 37, 16788-16801 (1998); Jeffrey et al., Nature, 376, 313-320 (1995); WIPO International Publication No. WO 97/34876; and WIPO International Publication No. WO 96/14843. These properties may be assessed, for example, by using one or more of the biological testing procedures set out in the examples below.

The examples and preparations provided below further illustrate and exemplify the dosage forms and methods of the present invention. It is to be understood that the scope of the present invention is not limited in any way by the scope of the following examples.

### EXAMPLE 1

The compound of formula 1 was tested for: (1) in vivo efficacy under several scheduling: sid, weekend dose holiday and intermittent dosing; (2) efficacy when combined with docetaxel in xenograft models; (3) in vitro eNOS and Akt phosphorylation in endothelial cells; (4) the concentration of Nitro Oxide and related products in cell culture and in vivo and (5) use of c-Kit signal in the whole blood cells as a potential biomarker for the compound.

Biological Testing; Enzyme Assays

The stimulation of cell proliferation by growth factors such as VEFG, FGF, and others is dependent upon their induction of autophosphorylation of each of their respective receptor's tyrosine kinases. Therefore, the ability of a protein kinase inhibitor to block cellular proliferation induced by these growth factors is directly correlated with its ability to block receptor autophosphorylation. To measure the protein kinase inhibition activity of the compounds, the following constructs were devised.

VEGF-R2 Construct for Assay: This construct determines the ability of a test compound to inhibit tyrosine kinase activity. A construct (VEGF-R2Δ50) of the cytosolic domain of human vascular endothelial growth factor receptor 2 (VEGF-R2) lacking the 50 central residues of the 68 residues of the kinase insert domain was expressed in a baculovirus/insect cell system. Of the 1356 residues of full-length VEGF-R2, VEGF-R2Δ50 contains residues 806-939 and 990-1171, and also one point mutation (E990V) within the kinase insert domain relative to wild-type VEGF-R2. Autophosphorylation of the purified construct was performed by incubation of the enzyme at a concentration of 4 μM in the presence of 3 mM ATP and 40 mM MgCl₂ in 100 mM HEPES, pH 7.5, containing 5% glycerol and 5 mM DTT, at 4° C. for 2 h. After autophosphorylation, this construct has been shown to possess catalytic activity essentially equivalent to the wild-type autophosphorylated kinase domain construct. See Parast et al., Biochemistry, 37, 16788-16801 (1998).

FGF-R1 Construct for Assay: The intracellular kinase domain of human FGF-R1 was expressed using the baculovirus vector expression system starting from the endogenous methionine residue 456 to glutamate 766, according to the residue numbering system of Mohammadi et al., Mol. Cell. Biol., 16, 977-989 (1996). In addition, the construct also has the following 3 amino acid substitutions: L457V, C488A, and C584S.

LCK Construct for Assay: The LCK tyrosine kinase was expressed in insect cells as an N-terminal deletion starting from amino acid residue 223 to the end of the protein at residue 509, with the following two amino acid substitutions at the N-terminus: P233M and C224D.

CHK-1 Construct for Assay: C-terminally His-tagged full-length human CHK-1 (FL-CHK-1) was expressed using the baculovirus/insect cell system. It contains 6 histidine residues (6×His-tag) at the C-terminus of the 476 amino acid human CHK-1. The protein was purified by conventional chromatographic techniques.

CDK2/Cyclin A Construct for Assay: CDK2 was purified using published methodology (Rosenblatt et al., J. Mol. Biol., 230, 1317-1319 (1993)) from insect cells that had been infected with a baculovirus expression vector. Cyclin A was purified from E. coli cells expressing full-length recombinant cyclin A, and a truncated cyclin A construct was generated by limited proteolysis and purified as described previously (Jeffrey et al., Nature, 376, 313-320 (1995)).

CDK4/Cyclin D Construct for Assay: A complex of human CDK4 and cyclin D3, or a complex of cyclin D1 and a fusion protein of human CDK4 and glutathione-S-transferase (GST-CDK4), was purified using traditional biochemical chromatographic techniques from insect cells that had been co-infected with the corresponding baculovirus expression vectors.

VEGF-R2 Assay: Coupled Spectrophotometric (FLVK-P) Assay

The production of ADP from ATP that accompanies phosphoryl transfer was coupled to oxidation of NADH using phosphoenolpyruvate (PEP) and a system having pyruvate kinase (PK) and lactic dehydrogenase (LDH). The oxidation of NADH was monitored by following the decrease of absorbance at 340 nm (e₃₄₀=6.22 cm⁻¹ mM⁻¹) using a Beckman DU 650 spectrophotometer. Assay conditions for phosphorylated VEGF-R2Δ50 (indicated as FLVK-P in the tables below) were the following: 1 mM PEP; 250 μM NADH; 50 units of LDH/mL; 20 units of PK/mL; 5 mM DTT; 5.1 mM poly(E₄Y₁); 1 mM ATP; and 25 mM MgCl₂ in 200 mM HEPES, pH 7.5. Assay conditions for unphosphorylated VEGF-R2Δ50 (indicated as FLVK in the tables) were the following: 1 mM PEP; 250 μM NADH; 50 units of LDH/mL; 20 units of PK/mL; 5 mM DTT; 20 mM poly(E₄Y₁); 3 mM ATP; and 60 mM MgCl₂ and 2 mM MnCl₂ in 200 mM HEPES, pH 7.5. Assays were initiated with 5 to 40 nM of enzyme. Kᵢ values were determined by measuring enzyme activity in the presence of varying concentrations of test compounds. The data were analyzed using Enzyme Kinetic and Kaleidagraph software.

ELISA Assay: Formation of phosphogastrin was monitored using biotinylated gastrin peptide (1-17) as substrate. Biotinylated phosphogastrin was immobilized using streptavidin coated 96-well microtiter plates followed by detection using anti-phosphotyrosine-antibody conjugated to horseradish peroxidase. The activity of horseradish peroxidase was monitored using 2,2′-azino-di-[3-ethylbenzathiazoline sulfonate(6)]diammonium salt (ABTS). Typical assay solutions contained: 2 μM biotinylated gastrin peptide; 5 mM DTT; 20 μM ATP; 26 mM MgCl₂; and 2 mM MnCl₂ in 200 mM HEPES, pH 7.5. The assay was initiated with 0.8 nM of phosphorylated VEGF-R2Δ50. Horseradish peroxidase activity was assayed using ABTS, 10 mM. The horseradish peroxidase reaction was quenched by addition of acid (H₂SO₄), followed by absorbance reading at 405 nm. Kᵢ values were determined by measuring enzyme activity in the presence of varying concentrations of test compounds. The data were analyzed using Enzyme Kinetic and Kaleidagraph software.

FGF-R Assay: The spectrophotometric assay was carried out as described above for VEGF-R2, except for the following changes in concentration: FGF-R=50 nM, ATP=2 mM, and poly(E4Y1)=15 mM.

LCK Assay: The spectrophotometric assay was carried out as described above for VEGF-R2, except for the following changes in concentration: LCK=60 nM, MgCl₂=0 mM, poly(E4Y1)=20 mM.

CHK-1 Assay: The production of ADP from ATP that accompanies phosphoryl transfer to the synthetic substrate peptide Syntide-2 (PLARTLSVAGLPGKK) was coupled to oxidation of NADH using phosphoenolpyruvate (PEP) through the actions of pyruvate kinase (PK) and lactic dehydrogenase (LDH). The oxidation of NADH was monitored by following the decrease of absorbance at 340 nm (ε340=6.22 cm⁻¹ mM⁻¹) using a HP8452 spectrophotometer. Typical reaction solutions contained: 4 mN PEP; 0.15 mM NADH; 28 units of LDH/mL; 16 units of PK/mL; 3 mM DTT; 0.125 mM Syntide-2; 0.15 mM ATP; 25 mM MgCl₂ in 50 mM TRIS, pH 7.5; and 400 mM NaCl. Assays were initiated with 10 nM of FL-CHK-1. Kᵢ values were determined by measuring initial enzyme activity in the presence of varying concentrations of test compounds. The data were analyzed using Enzyme Kinetic and Kaleidagraph software.

HUVEC Proliferation Assay: This assay determines the ability of a test compound to inhibit the growth factor-stimulated proliferation of human umbilical vein endothelial cells (“HUVEC”). HUVEC cells (passage 3-4, Clonetics, Corp.) were thawed into EGM2 culture medium (Clonetics Corp) in T75 flasks. Fresh EGM2 medium was added to the flasks 24 hours later. Four or five days later, cells were exposed to another culture medium (F12K medium supplemented with 10% fetal bovine serum (FBS), 60 μg/mL endothelial cell growth supplement (ECGS), and 10 μg/m heparin). Exponentially-growing HUVEC cells were used in experiments thereafter. Ten to twelve thousand HUVEC cells were plated in 96-well dishes in 100 μL of rich, culture medium (described above). The cells were allowed to attach for 24 hours in this medium. The medium was then removed by aspiration and 115 μL of starvation media (F12K+1% FBS) was added to each well. After 18 hours, 15 μL of test agent dissolved in 1% DMSO in starvation medium or this vehicle alone was added into each treatment well; the final DMSO concentration was 0.1%. One hour later, 20 ul of 150 ng/mL hrVEGF₁₆₅ in starvation media was added to all wells except those containing untreated controls; the final VEGF concentration was 20 ng/mL. Cellular proliferation was quantified 72 hours later by MTT dye reduction, at which time cells were exposed for 4-5 hours MTT (Promega Corp.). Dye reduction was stopped by addition of a stop solution (Promega Corp.) and absorbance at 570 and 630 nm was determined on a 96-well spectrophotometer plate reader.

Cancer Cell Proliferation (MV522) Assay: To determine the whether a protein kinases inhibitor should have therapeutic usefulness in blocking angiogenesis for treating cancer, it is important to demonstrate the inhibitor does not non-specifically block cellular proliferation in cells that do not express the kinase receptor. This is done by performing proliferation assays using cancer cells. The protocol for assessing cellular proliferation in cancer cells is similar to that used for assessments in HUVEC cells. Two thousand lung cancer cells (line MV522, acquired from UCSD) were seeded in growth media (RPMI1640 medium supplemented with 2 mM glutamine and 10% FBS). Cells are allowed to attach for 1 day prior to addition of test agents and/or vehicles. Cells are treated simultaneously with the same test agents used in the HUVEC assay. Cellular proliferation is quantified by MTT dye reduction assay 72 hours after exposure to test agents.

C-Kit potency determination: NCI-H526 (ATCC) cells were used for determining potency against c-Kit by the inhibitor. The cells were grown to sub-confluency and incubated in starvation media for 18 hours. The inhibitor was added and the cells were incubated for 45 min at 37° C. in the presence of 2.3% albumin and 1 mM Na₃VO₄ (Sigma). SCF, the c-Kit growth factor was added to the culture at a final concentration of 50 ng/mL. Five minutes later the cells were rinsed 2× with cold PBS and lysed with lysis buffer (50 mM Tris, 150 mM NaCl, 1 mM PMSF, 1% NP40, 1 mM Na₃VO₄ and a protease inhibitor cocktail). Immunoprecipitation was performed using 1 mg total protein from each lysate, incubating over night at 4° with 4 μg/mL CD117 ab-3 (K45, Neomarkers). The antibody complex was conjugated to protein A beads the following morning. SDS PAGE and Western Blot analysis was conducted using anti phosphotyrosine antibody 4G10 (Upstate Biotechnology) for phosphorylated receptors, or anti-c-Kit receptor antibody sc-1493 (C-14, Santa Cruz) at 1:1000. The blots were visualized by the chemiluminescent reagents ECL Plus. A phosphorimager (Storm 846, Molecular Dynamics) was used for the quantification of the signals in the blots.

The reduction of c-kit positive cell population in total peripheral blood cells of an animal and mammal may be used as a biomarker for activity of the compound of formula 1.

ENOS and Akt phosphorylation measurement: HUVEC (Clonetics) were used for determining potency against eNOS and Akt by the inhibitor. The cells were grown to sub-confluency and incubated in starvation media for 18 hours. The inhibitor was added and the cells were incubated for 45 min at 37° C. in the presence of 2.3% albumin and 1 mM Na₃VO₄ (Sigma). VEGF was added to the culture medium at 50 ng/mL. Five minutes later the cells were rinsed 2× with cold PBS and lysed with lysis buffer (50 mM Tris, 150 mM NaCl, 1 mM PMSF, 1% NP40, 1 mM Na₃VO₄ and a protease inhibitor cocktail). A total protein of 30-40 ug was analyzed by the Western method. eNOS and Akt Phosphorylation was assessed by using: Phospho-eNOS (Ser 1177) #9571 or Phospho-Akt (Ser 473) #9271 antibodies (both from Cell signaling). Protein detection was achieved by using: NOS3 (C-20) sc-654 (Santa Cruz) or Akt antibody #9272 (Cell Signaling). All require an anti rabbit HRP linked secondary antibody used at 1:3000. The blots were visualized by the chemiluminescent substrate Super Signal West Dura (Pierce). An Alpha Imager 8800 from Alpha Innotech was used for the quantification of the signals in the blots.

Mouse PK Assay: The pharmacokinetics (e.g., absorption and elimination) of drugs in mice were analyzed using the following experiment. Test compounds were formulated as a suspension in a 0.5% CMC vehicle or as a solution in a 30:70 (PEG400:acidified H₂O) vehicle. This suspension or solution was administered orally (p.o.) or intraperitoneally (i.p.) to the C3H female mice (n=4). Blood samples were collected via an orbital bleed at time points: 0 hour (pre-dose), 0.5 hr, 1.0 hr, 2.0 hr, and 4.0 hr post dose. Plasma was obtained from each sample by centrifugation at 2500 rpm for 5 min. Test compound was extracted from the plasma by an organic protein precipitation method. For each time bleed 50 μL of plasma was combined with 1.0 mL of acetonitrile, vortexed for 2 min. and then spun at 4000 rpm for 15 min. to precipitate the protein and extract out the test compound. Next, the acetonitrile supernatant (the extract containing test compound) was poured into new test tubes and evaporated on a hot plate (25° C.) under a steam of N₂ gas. To each tube containing the dried test compound extract 125 μL of mobile phase (60:40, 0.025 M NH₄H₂PO₄+2.5 mL/L TEA:acetonitrile) was added. The test compound was resuspended in the mobile phase by vortexing and more protein was removed by centrifugation at 4000 rpm for 5 min. Each sample was poured into an HPLC vial for test Compound Analysis on an Hewlett Packard 1100 series HPLC with UV detection. From each sample, 95 μL was injected onto a Phenomenex-Prodigy reverse phase C-18, 150×3.2 mm column and eluted with a 45-50% acetonitrile gradient run over 10 min. Test-compound plasma concentrations (μg/mL) were determined by a comparison to standard curve (peak area vs. conc. μg/mL) using known concentrations of test compound extracted from plasma samples in the manner described above. Along with the standards and unknowns, three groups (n=4) of quality controls (0.25 μg/mL, 1.5 μg/mL, and 7.5 μg/mL) were run to insure the consistency of the analysis. The standard curve had an R2>0.99 and the quality controls were all within 10% of their expected values. The quantitated test samples were plotted for visual display using Kaleidagraph software and their pharmacokinetic parameters were determined using WIN NONLIN software.

Human Liver Microsome (HLM) Assay: Compound metabolism in human liver microsomes was measured by LC-MS analytical assay procedures as follows. First, human liver microsomes (HLM) were thawed and diluted to 5 mg/mL with cold 100 mM potassium phosphate (KPO4) buffer. Appropriate amounts of KPO4 buffer, NADPH-regenerating solution (containing B-NADP, glucose-6-phosphate, glucose-6-phosphate dehydrogenase, and MgCl₂), and HLM were preincubated in 13×100 mm glass tubes at 37 C for 10 min. (3 tubes per test compound—triplicate). Test compound (5 μM final) was added to each tube to initiate reaction and was mixed by gentle vortexing, followed by incubation at 37 C. At t=0, 2 h, a 250-uL sample was removed from each incubation tube to separate 12×75 mm glass tubes containing 1 mL ice-cold acetonitrile with 0.05 μM reserpine. Samples were centrifuged at 4000 rpm for 20 min. to precipitate proteins and salt (Beckman Allegra 6KR, S/N ALK98D06, #634). Supernatant was transferred to new 12×75 mm glass tubes and evaporated by Speed-Vac centrifugal vacuum evaporator. Samples were reconstituted in 200 μL 0.1% formic acid/acetonitrile (90/10) and vortexed vigorously to dissolve. The samples were then transferred to separate polypropylene microcentrifuge tubes and centrifuged at 14000×g for 10 min. (Fisher Micro 14, S/N M0017580). For each replicate (#1-3) at each timepoint (0 and 2 h), an aliquot sample of each test compound was combined into a single HPLC vial insert (6 total samples) for LC-MS analysis, which is described below.

The combined compound samples were injected into the LC-MS system, composed of a Hewlett-Packard HP1100 diode array HPLC and a Micromass Quattro II triple quadruple mass spectrometer operating in positive electrospray SIR mode (programmed to scan specifically for the molecular ion of each test compound. Each test compound peak was integrated at each timepoint. For each compound, peak area at each timepoint (n=3) was averaged, and this mean peak area at 2 h was divided by the average peak area at time 0 hour to obtain the percent test compound remaining at 2 h.

In Vitro HUVEC Apoptosis Assays

Quantification of Apoptosis by ELISA: Apoptosis of HUVEC cells was measured using Cell Death Detection Elisa PLUS (catalog #1775425, Roche Biochemicals, Mannheim, Germany) that quantifies cytoplasmic histone-associated DNA fragments in cell lysates. The procedure was performed with minor modifications to the manufacture's instructions. Briefly, Starved HUVEC cells were treated with various concentrations of Compound A in the presence of VEGF (20 ng/mL). The cytosolic fraction of the cells at various time points was collected and used as an antigen source in a sandwich ELISA with a primary anti-histone mAb coated to the microtiter plate and a secondary anti-DNA mAb coupled to peroxidase. The number of apoptotic cells was determined by adding chromogenic peroxidase substrate and measuring the absorption with a spectrophotometer at 405 nm (reference wavelength 490 nm).

Visualization of Apoptosis by TUNEL: In situ detection of apoptotic cell was carried out using the TdT-mediated dUTP nick end labeling (TUNEL) technique. Briefly HUVEC cells grown in 8 well Lab-Tek chamber slides were starved O/N and then treated for 6 hours with various concentrations of Compound A. The cells were then fixed in 4% Paraformaldehyde, permeablized with Triton X-100 and incubated for 1 hour in a mixture of terminal transferase and nucleotides including Fluorescein-dUTP (Deadend Fluorometric TUNEL system, Promega, catalog # G3250) in accordance with the manufacturer's instructions. The cells were counterstained with Propidium iodide (PI) solution. Positively stained Fluorescein and PI labeled cells were visualized and photographed by fluorescence microscopy.

PDGF mediated Cell Migration Assay: U87MG cells were used in this assay. Six well plates are pre-incubated overnight with 0.5 ng/mL Fibronectin. The following day U87MG cells are plated in each well and allowed to grow to confluence. The cells were incubated overnight with starvation media containing 0.1% FBS. A ˜1 cm scratch was made using a pipette tip and the cells washed with the starvation media. The plates were then incubated with 0.5 ng/mL Fibronectin for 1 hour and then washed again. The experimental media containing 100 ng/Ll rhPDGF BB and Compound A in the starvation media was introduced. Cells were photographed between 0 and 15 hour and the migration was visualized.

Cellular VEGFR-2 and Downstream Molecule Phosphorylation Assay: HUVECs (Clonetics) were cultured to sub-confluency and incubated in starvation media (F12K plus 0.1% FBS) for 18 hours. Compound A was added to the cells in the presence of 2.3% albumin and 1 mM Na₃VO₄ (Sigma). Forty-five minutes later, VEGF was added to the culture with a final concentration of 50 ng/mL. Five minutes later the cells were rinsed with cold PBS and lysed with lysis buffer (50 mM Tris, 150 mM NaCl, 1 mM PMSF, 1% NP40, 1 mM Na₃VO₄ and a protease inhibitor cocktail). One milligram of total proteins from lysate was immunoprecipitated using anti-Flk-1 C-1158 (Santa Cruz). The antibody complex was conjugated to protein A beads and SDS PAGE/Western analysis was conducted. phosphorylated VEGFR-2 and the protein was detected by the anti phosphotyrosine antibody 4G10 (Upstate Biotechnology) and anti-Flk-1 C-20 (Santa Cruz), respectively. For eNOS and Akt, the cells were treated the same as above. Western analyses were performed using a total of 30-40 μg proteins. eNOS and Akt phosphorylation was probed by using Phospho-eNOS (Ser 1177, #9571) or Phospho-Akt (Ser 473, #9271) antibodies (Cell Signaling). Proteins were assessed by using NOS3 C-20 (sc-654, Santa Cruz) or Akt antibody #9272 (Cell Signaling). HRP linked anti-rabbit IgG was used as the secondary antibody. All blots were visualized by the chemiluminescent substrate Super Signal West Dura (Pierce). The signal was quantified using an Alpha Imager 8800 from Alpha Innotech.

Washout Experiments: HUVEC cells were treated as described above. After incubation with Compound A (10 nM) for 45 min and stimulated with VEGF (50 ng/mL) for 5 min, the supernatant was removed, washed and replace with the starvation media containing VEGF and Na₃VO₄. The cells were further incubated for desired length of time before lysed and processed using immunoprecipitation and Western for phosphorylated and total VEGFR-2 (see above). In another experiment, the cells were treated with VEGF for the entire length of time as above and VEGFR-2 phosphorylation and total VEGFR-2 at desired time points were assessed similarly. Signals during washout were quantified by densitometry. Intensities of maximum stimulation (5 min) from each experiment was normalized to each other and the intensity of phospho-VEGFR-2 at each time point was compared across the two experiments, which allowed to determine VEGFR-2 phosphorylation recovery relative to cells that were untreated but VEGF-stimulated.

Tumor Models: For the human MV522 (colon carcinoma) and MDA-MB-231 (breast carcinoma) models, athymic mice (n=8˜12) were implanted (s.c.) with 5×10⁶ cells/site; For the murine Lewis Lung carcinoma model, tumor fragments (1-2 mm²) were trocar-implanted in the right flank of B6D2 μl mice. Dosing usually started on day-7 (MV522) or when average tumor size reached 150-200 mm³ (MDA-MB-231).

The compound of formula 1 was formulated in 0.5% CMC/H₂O and administered PO, BID. Docetaxel was formulated in 7% EtOH/3% Polysorbate/90% H₂O and was dosed weekly, intravenously. Treatment usually lasted for 3-4 weeks. The geometric length and width of the tumor was measured three times per week using an electronic caliper. Tumor volume was calculated as a product of 0.4×[Length×(Width)²]. Data were reported as mean±SEM. At end of studies, tumors and tissues were resected, weighed and collected for analysis. Plasma was collected for analysis of drug concentration.

Results are shown in Tables 1-3.

TABLE 1
Potency and Selectivity of Compound 1

| | Enzymatic Activity, | Receptor Phosphorylation, |
|---|---|---|
| Target | Kᵢ (nM) | IC₅₀ (nM)^{a} |
| VEGFR-2 (KDR) | 1.1 | 0.25 |
| VEGFR-1 (FIt-1) | 8.3 | 1.2^{b} |
| VEGFR-3 (FIt-4) | nd | 0.29 |
| PDGFR-β | 1.3 | 2.5 |
| c-Kit | nd | 2 |
| FGFR-1 | 56 | 218 |

^{a}measured by cell proliferation assays;
^{b}measured in the presence of 2.3% albumin by IP/IB;
nd: Not determined.
Other enzyme screened but were above limit for Kᵢ calculation are: cMet, LCK, c-Src, FAK, Pyk2, IRL, BTK, CDK1, CDK2, CDK4, PKA, PKC, PLK and Chk1.

TABLE 2
Study design for the co-administration of Compound 1 and
docetaxel in the MDA-MB-231 human breast cancer model.

| Compound 1 (mg/kg)^{a} | Docetaxel^{b} | Dose Selection Rationale |
|---|---|---|
| 25 | 0 | ED₉₀ |
| 5 | 0 | ED₅₀ |
| 1 | 0 | low dose |
| 0 | 20 | 70% MTD for mouse |
| 0 | 10 | calc. equiv. human MTD |
| 0 | 2 | low dose |
| 25 | 20 | tolerance and DDI |
| 5 | 10 | additivity and DDI |
| 5 | 2 | additivity and DDI |
| 1 | 10 | additivity and DDI |
| 1 | 2 | additivity and DDI |

^{a}po, bid, daily;
^{b}iv, once/week

TABLE 3
Combination therapy of docetaxel and Compound 1 produced
greater anti-tumor activity in MDA-MB-231 xenograft model.

| Compound 1 | | | |
|---|---|---|---|
| (mg/kg)^{a} | Docetaxel^{b} | PR* | CR** |
| 0 | 0 | 0 | 0 |
| 25 | 0 | 3 | 0 |
| 5 | 0 | 3 | 0 |
| 1 | 0 | 0 | 0 |
| 0 | 20 | 4 | 0 |
| 0 | 10 | 6 | 0 |
| 0 | 2 | 0 | 0 |
| 25 | 20 | 12 | 0 |
| 5 | 10 | 10 | 2 |
| 1 | 10 | 7 | 2 |
| 5 | 2 | 0 | 0 |
| 1 | 2 | 0 | 0 |

^{a}po, bid, daily;
^{b}iv, once/week

The combination groups demonstrate the increased incidences of complete and partial tumor regression. Tumor growth rate was reduced to a greater degree when the agents were combined. The combination treatment was equally well tolerated than the single agents alone.

### EXAMPLE 2

The compound of formula 1, 6-[2-(methylcarbamoyl)phenylsulfanyl]-3-E-[2-(pyridin-2-yl) ethenyl]indazole, was administered in varying doses to patients with solid tumors. Thirty patients (13 male, 17 female) were treated using the compound of formula 1 in an oral dosage, tablet form, on a BID or QD schedule. Cycles were 28 days each. The specific tumor diagnoses were breast (11), thyroid (5), renal cell (5), lung (4) and other (5). Pharmacokinetic data were measured by liquid chromatography-tandem mass spectrometry (LC-MS/MS). Blood samples were taken on day 15 of the cycle at times of ½ hour, 1 hour, 2 hours, 4 hours, 8 hours and 12 hours from the time of administration.

Pharmacokinetic results (day 15 mean values) are shown in Table 4. The patients were not fasted unless otherwise indicated. The numbers in parentheses are the coefficient of variation expressed as a percentage. In the Table, Cₘₐₓ is the maximum observed blood plasma concentration of the compound of formula 1, AUC (0-24) is the 24-hour AUC blood plasma concentration, and T_{1/2} is the half-life as determined from a concentration versus time plot. The entry “# patients with PK” indicates the number of patients for whom pharmacokinetic data were obtained.

TABLE 4

| Dose | | | | |
|---|---|---|---|---|
| Schedule and | # patients/ | Cₘₐₓ | AUC (0-24) | T_{1/2} |
| Amount | # patients with PK | (ng/mL) | (ng · hr/mL) | (hr) |
| 5 mg BID | 6/6 | 27.1 (36) | 257 (39) | 2.2 (16) |
| 5 mg BID | 8/6 | 54.5 (48) | 311 (76) | 2.7 (39) |
| fasted | | | | |
| 15 mg QD | 6/6 | 78.6 (54) | 797 (96) | 3.5 (46) |
| 20 mg BID | 4/3 | 129.4 (86) | 1524 (87) | 3.1 (51) |

In addition, patients in the first cohort (n=6) received individualized doses ranging from 10 mg QD to 30 mg BID (PK not shown). Plasma exposures were higher (about 49%) and intra-patient variability was reduced, in the fasted versus fed state. The maximum tolerated dose (MTD) at the present time has been determined to be 5 mg BID fasted. Dose-limiting toxicities (DLTs) at doses greater than the MTD were hypertension (HTN), seizure, elevated liver function tests, pancreatitis, apnea and stomatitis. In addition, 2 responding patients with NSCLC had fatal hemoptysis, one 3 weeks after stopping the compound treatment. Non-dose-limiting proteinuria was also observed. At doses less than or equal to the MTD, the DLT was limited to grade 2 stomatitis in 1 patient. Non-dose-limiting HTN was observed in 7/14 patients and was managed by conventional hypertensive medications. Two durable partial responses by RECIST criteria were observed (in renal call and adenoid cystic tumor of the maxillary sinus) and stable disease lasting greater than or equal to 4 month (range 4-13+ months) in 5 patients of this heavily pretreated population. Using dceMRI, preliminary analysis of 21 patients was performed to measure vascular effected induced by the compound of formula 1 at baseline, and on days 2, 28 and 56. The percentage change in mean K^{trans} (P. S. Tofts, G. Brix, D. L. Buckley, J. L. Evelhoch, E. Henderson, M. V. Knopp, H. B. W. Larsson, T. Lee, N. A. Mayr, G. J. M. Parker, R. E. Port, J. Taylor and R. M. Weisskoff, Estimating Kinetic Parameters from Dynamic Contrast-Enhanced T₁-Weighted MRI of a Diffusable Tracer: Standardized Quantities and Symbols, Journal of Magnetic Resonance Imaging, 10:223-232 (1999)) and initial area under the contrast intensity X time curve (IAUC) was computed for each index tumor (n=1-4 per patient). A tumor vascular response was defined as greater than or equal to 50% decrease in baseline parameter values to day 2. Acute (day 2) decreases in tumor vascular response (greater than or equal to 50% decrease in K^{trans} and IAUC) were observed in 6/18 evaluable patients, and 11/18 demonstrated a greater than or equal to 40% decrease in both K^{trans} and IAUC. Due to technical issues with the scans, 3/21 image sets were not evaluable. This example shows that the compound of formula 1 is a highly active agent as manifested by clinical response and acute tumor vascular changes.

### EXAMPLE 3

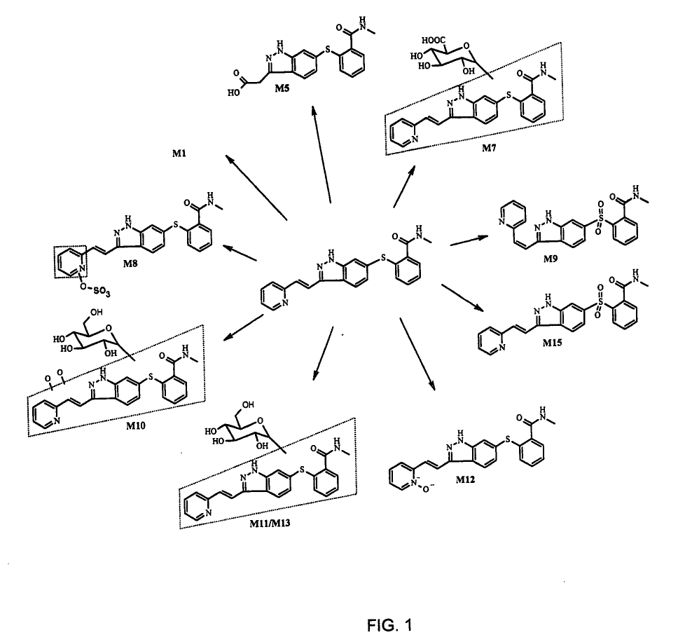

Following oral administration of a 30 mg free base/kg dose of [¹⁴C]-labeled compound of formula 1 to intact or bile duct-cannulated beagle dogs, extensive metabolism was observed. Biotransformation pathways included oxygenation (mono- or di-), glucuronidation, glucosylation, and oxygenation followed by either sulfation or glucosylation. FIG. 1 shows the identified metabolites. In plasma, M12 (an N-oxide) is the only metabolite detectable. In urine, M5 (a depyridinyl carboxylic acid) is the major metabolite. The major biliary metabolites include M8 (a sulfate) and M12. The chemical structure of the major fecal metabolite M1 remains unknown.

Excretion patterns for [¹⁴C]-derived radioactivity in beagle dogs following a single oral dose of the compound were similar for males and females, with radioactivity excreted primarily via feces. Mean recoveries for intact males were 85.5% in feces and 5.3% in urine, compared to recoveries of 80.9% in feces and 7.0% in urine for intact females. Bile duct-cannulated male dogs excreted a relatively small fraction of radioactivity in bile (8.3% recovery), with additional radioactivity recovered in feces (52.7%) and urine (11.3%). The combined total of urinary and biliary radioactivity from bile duct-cannulated dogs suggests that approximately 20% of administered radioactivity underwent gastrointestinal absorption. The total mean recoveries in all samples were 92.4% and 92.6% for intact males and females, respectively, and 89.6% for bile duct-cannulated males. All metabolite profiling and structure elucidation were performed using HPLC coupled in-line with radio-HPLC detector (β-RAM) and MS detection with electrospray (ESI) and atmospheric pressure chemical ionization (APCI) sources in positive or negative mode.

### EXAMPLE 4

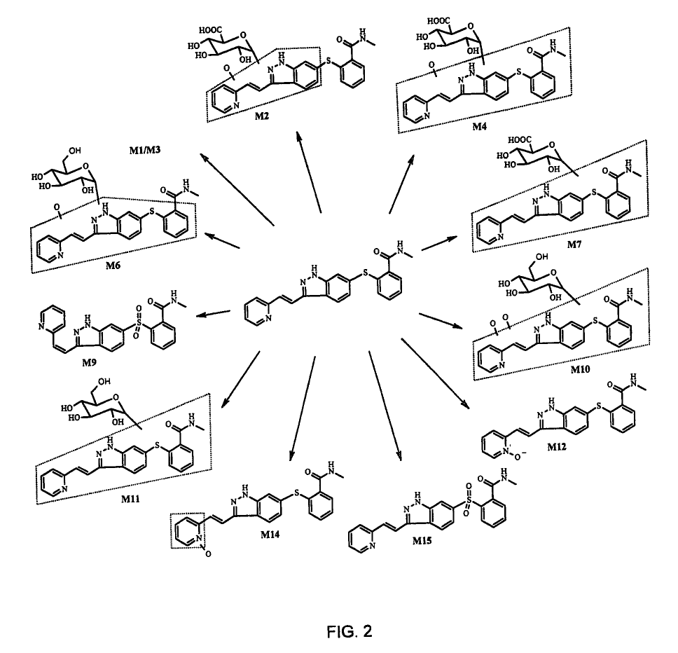

The compound of formula 1 undergoes extensive metabolism in CD-1 mice following single oral administration of the [¹⁴C]-labeled compound. A low percentage of unchanged drug was recovered in urine and feces, and a variety of phase I and phase II metabolites were observed. Biotransformation pathways included oxygenation (mono- or di-), glucuronidation, glucosylation and oxygenation followed by either glucuronidation or glucosylation. The metabolites identified are shown in FIG. 2. In plasma, unchanged drug and M12 (an N-oxide) represented the two major components. M7 (a glucuronide) represented the most significant metabolite in both urine and feces.

The majority of [¹⁴C]-derived radioactivity was recovered in feces following a single oral administration of 50 mg free base/kg dose of [¹⁴C]AG-013736 to male CD-1 mice. Mean (n=2) recoveries of the radioactivity (% of dose) at 48 hours postdose were 65.8% in feces and 12.7% in urine. The rate of elimination of radioactivity in excreta was rapid with ˜72% of the dose recovered within 24 hours postdose. Radioactivity profiling and structure characterization of metabolites was performed using LC-RAM-MS methods.

In addition to the metabolites shown in FIGS. 1 and 2, other known metabolites include the active des-methyl metabolite shown in formula 1a.

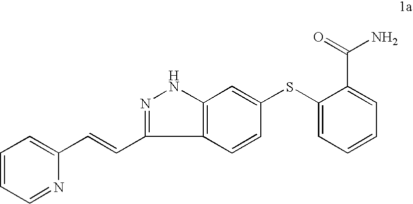

### EXAMPLE 5

Angiogenesis was assessed by measuring tumor microvessel density (MVD) using immunohistochemistry. Frozen tumor sections were stained for vessel surface marker CD-31 and the amount of vessels in several fields of the tissue section were quantified manually. Studies demonstrated that PO BID administration of the compound of formula 1 for 2 to 3 weeks reduced the number of blood vessels in treated tumors by 70% compared with the control tumors. This decrease of microvessel density after treatment was observed across all tumor models used, including the LLC, MV522, and M24met. When delivered continuously via an osmotic Alzet pump in the LLC tumor model, the compound of formula 1 produced a significant growth inhibition. Data from 3 studies indicated that the maximum tumor growth inhibition that can be achieved by this class of agent in the LLC model was 78%. At plasma concentrations as low as 55±17 ng/mL (N=3), 90% of maximum growth inhibition was achieved. This concentration was designated as the biologically active concentration (BAC). The 50% maximum growth inhibition was associated with a plasma concentration of 28±11 ng/mL (N=3). This concentration was designated as the minimal efficacious concentration (MEC). In 1 study group, 70% of MGI produced by continuous infusion of the compound was associated with an AUC(0-24) of 574 ng·hr/mL, whereas in the same study an AUC(0-24) of 720 ng h/mL after PO BID dosing resulted in a 40% maximal growth inhibition (MGI. These results suggest that antitumor efficacy seen in this model is driven by trough concentration and that in mice, a continuous low concentration of the compound may be sufficient to produce maximal antitumor efficacy.

The compound of formula 1 was efficacious as a single agent in the human breast carcinoma xenograft model MDA-MB-231. In preparation for an efficacy study with the combination of the compound of formula 1 and docetaxel in this model, a preliminary study in naïve nude mice was conducted to determine the effect of potential drug-drug interactions on PK and tolerability. Following IV administration of 15 or 30 mg/kg docetaxel once per week for 3 weeks, a decrease in body weight (7% and 11%, respectively) compared with control was identified in docetaxel-treated animals. No difference in body weight was noted between animals treated with docetaxel alone and those given the combination of docetaxel and the compound of formula 1 (30 mg/kg/day for 16 days; PO). Docetaxel administration did not affect the AUC of the compound of formula 1, whereas Cₘₐₓ values of AG-013736 were reduced significantly in the combination groups compared with the compound of formula 1 alone.

Histologic examination of selected tissues (liver, kidneys, heart, spleen, stomach, small and large intestines, ovaries, sternum, joint) revealed no target organ effects in mice treated with the compound of formula 1 as a single agent in this study. Changes noted in docetaxel-treated mice included ovarian follicular necrosis and minimal to mild bone marrow hypocellularity. The combined treatment of the compound of formula 1 and docetaxel did not exacerbate the effect of docetaxel on the ovary, but an increased intensity of bone marrow hypocellularity was noted (minimal to moderate) in animals given the compound of formula 1/docetaxel combination. In addition, bone marrow hemorrhage was observed in combination-treated animals, likely a secondary effect of the increased intensity of hypocellularity.

the compound of formula 1 and docetaxel were combined for efficacy assessment in the MDA-MB-231 tumor model. The compound of formula 1 alone (25, 5, and 1 mg/kg, PO, BID, given for 3 weeks) resulted in dose-dependent tumor growth inhibition. Docetaxel alone (IV, weekly) at 20 and 10 mg/kg, but not 2 mg/kg, was also efficacious. It appeared that there might be a beneficial therapeutic interaction between the compound of formula 1 and docetaxel. This benefit was more evident when combining the agents at both the high and middle doses. The incidences of partial regression (16% to 97% reduction in tumor size) and complete response in the high- and middle-dose combination arms were much greater than those in the groups of individual agent alone at the same doses. Due to limited groups and relatively short time frame of the study, this is not a definitive finding. The compound of formula 1 was well-tolerated at all doses. There was a 3% to 7% decline of the average body weight in the high-dose combination group (25 mg/kg compound 1 and 20 mg/kg docetaxel) after the third dose of the chemotherapeutic agent, compared with all the other groups. Pharmacokinetic analysis demonstrated that the AUC values of the compound of formula 1 were not affected in the presence of docetaxel, but values of Cₘₐₓ were reduced significantly in the combination groups compared with the compound of formula 1 alone group.

Anti-tumor efficacy of the compound of formula 1 in combination with docetaxel was investigated in the LLC model. The LLC model is highly resistant to docetaxel. At the reported MTD (30 mg/kg weekly dose, iv) little tumor growth delay (TGD) was seen with the cytotoxic agent (TGD=3.2 days). All mice were euthanized within 28 days of experiment due to large primary tumors. In contrast, single agent compound of formula 1 generated dose-dependent and statistically significant TGD (13.4 days at 10 mg/kg and 15.4 days at 30 mg/kg, PO, BID). However, the agent only delayed, but didn't stop, metastasis to the lung. The TGD (20.4 days) of the high dose combination group, but not the low dose combination group (TGD=15.2 days), was statistically different from either of the single agents alone (P=0.0079 and P=0.254, respectively). More animals (3/10) reached objective end point in the high dose combination group, but not in the low dose combination group. In conclusion, high dose combination therapy of the compound of formula 1 and docetaxel can generate greater delay of primary tumor growth and metastasis than either monotherapy alone, but it does not result in a complete cure.

One study using the MV522 tumor model demonstrated that a single daily (QD) 60 mg/kg PO dose of the compound of formula 1 resulted in a similar tumor growth inhibition effect as did 30 mg/kg PO, BID (p=0.154). In addition, antitumor efficacy did not appear to be compromised when dosed PO, BID at 30 mg/kg for 5 consecutive days followed by 2 dosing holidays, compared with the daily PO BID using the same dose concentration (p=0.223). These results suggest that in this nonclinical tumor model, it might be possible to give the compound of formula 1 with either QD or certain interim dosing scheduling and expect to achieve significant antitumor efficacy.

The amount of time of receptor inhibition and concentrations of the compound of formula 1 required to produce anti-tumor efficacy in the MV522 xenograft model were investigated. The results showed that with PO dosing (QD or BID), an approximately 24-hour daily exposure above the EC₅₀ (5 ng/mL) was necessary for a ≧50% antitumor efficacy. A minimum of 4-hour daily exposure at plasma concentration of ≧40-60 ng/mL was necessary in order to achieve a 90% tumor growth inhibition. An exposure beyond the above threshold did not warrant additional efficacy. There was a similar body weight loss in either the BID or the QD group; both were under 5%. Thus, given the appropriate dose and time of exposure, the QD regimen may be as effective as the BID regimen.

It was also demonstrated that continuous exposure via the Alzet pumps generated greater antitumor efficacy by the compound of formula 1 as compared with regular periodic dosing. Delivery by the pumps at 10 mg/mL produced a constant average systematic exposure of 30 ng/mL, which resulted in tumor stasis. In contrast, saturating doses (PO, BID), which yielded plasma concentrations of the compound of formula 1 above projected EC₉₀, could only generate tumor growth delay. Thus, continuous systematic exposure of the compound of formula 1 appeared to be more effective than the twice daily oral dosing regimen in treating the tumor.

Anti-tumor efficacy of the compound of formula 1 using an intermittent dosing regimen was also studied. The treatment groups were as follows: daily dosing vehicle, intermittent vehicle, daily dose of 30 mg/kg (BID), and an intermittent dose of 30 mg/kg. The intermittent dosing schedule was as follows: Cycle-1 (Days 12˜18—dosing on and Days 19˜28—dosing off), and Cycle 2 (Days 29˜36—dosing on and Days 37˜44—dosing off). Dosing started when the average tumor size was 250 mm³; all were given AG-013736 (PO, BID). Overall, there was a significant difference between the intermittent and daily BID dosing, with the continual, daily dosing regimen being more effective in generating growth delay. For the intermittently dosed drug group, tumors regained normal growth rate within 3-4 days after dosing was stopped. However, tumor growth inhibition resumed within 2 days of the Cycle-2 dosing. As expected, no regression was seen in any of the groups.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

We claim:

1. A dosage form for administration to a mammal, the dosage form comprising a compound of formula 1: 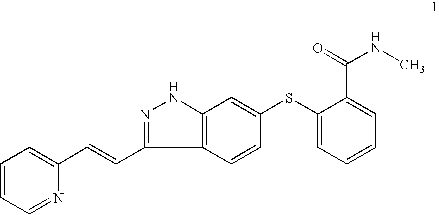
a pharmaceutically acceptable salt, solvate or prodrug thereof, or a mixture thereof, in an amount effective to provide a 24-hour AUC blood plasma value of no more than 4500 ng·hr/mL of the compound of formula 1 or active metabolites thereof, after administration to the mammal.

2. The dosage form of claim 1, wherein the 24-hour AUC blood plasma value is from 25 to 4500 ng·hr/mL.

3. The dosage form of claim 1, wherein the 24-hour AUC blood plasma value is from 50 to 2500 ng·hr/mL.

4. The dosage form of claim 1, wherein the 24-hour AUC blood plasma value is from 75 to 1000 ng·hr/mL.

5. The dosage form of claim 1, wherein the 24-hour AUC blood plasma value is from 100 to 800 ng·hr/mL.

6. The dosage form of claim 1, wherein the dosage form is an oral dosage form.

7. The dosage form of claim 1, wherein the dosage form is a tablet or a capsule.

8. A dosage form comprising a compound of formula 1: 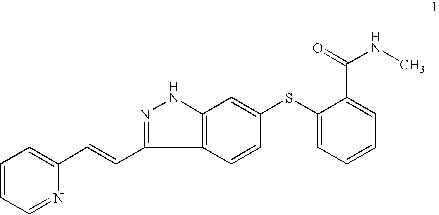
a pharmaceutically acceptable salt, solvate or prodrug thereof, or a mixture thereof, in an amount of no more than 30 mg.

9. The dosage form of claim 8, wherein the amount is from 0.5 to 30 mg.

10. The dosage form of claim 8, wherein the amount is from 1 to 20 mg.

11 The dosage form of claim 8, wherein the amount is from 1.5 to 15 mg.

12. The dosage form of claim 8, wherein the amount is from 2 to 10 mg.

13. The dosage form of claim 8, wherein the amount is from 2.5 to 8 mg.

14. The dosage form of claim 8, wherein the amount is from 3 to 7 mg.

15. The dosage form of claim 8, wherein the dosage form is an oral dosage form.

16. The dosage form of claim 8, wherein the dosage form is a tablet or capsule.

17. A method of treating abnormal cell growth in a mammal, the method comprising administering to the mammal a compound of formula 1: 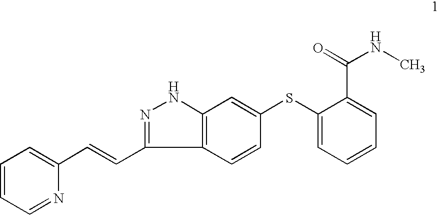
a pharmaceutically acceptable salt, solvate or prodrug thereof, or a mixture thereof, in an amount effective to provide a 24-hour AUC blood plasma value of no more than 4500 ng·hr/mL of the compound of formula 1 or active metabolites thereof, after administration to the mammal.

18. The method of claim 17, wherein the 24-hour AUC blood plasma value is from 25 to 4500 ng·hr/mL.

19. The method of claim 17, wherein the 24-hour AUC blood plasma value is from 50 to 2500 ng·hr/mL.

20. The method of claim 17, wherein the 24-hour AUC blood plasma value is from 75 to 1000 ng·hr/mL.

21. The method of claim 17, wherein the 24-hour AUC blood plasma value is from 100 to 800 ng·hr/mL.

22. The method of claim 17, wherein the compound is administered orally.

23. The method of claim 17, wherein the compound is administered at a dosage frequency of at least once per day.

24. The method of claim 17, wherein the compound is administered at a dosage frequency of at least twice per day.

25. The method of claim 17, wherein the mammal fasts for at least two hours prior to the step of administering.

26. The method of claim 17, wherein the mammal fasts for at least two hours after the step of administering.

27. The method of claim 17, wherein the mammal fasts for at least two hours prior to the step of administering and at least two after the step of administering.

28. The method of claim 17, wherein the abnormal cell growth is cancer.

29. The method of claim 28, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

30. The method of claim 17, wherein the method further comprises co-administering an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, anti-androgens and mixtures thereof.

31. The method of claim 30, wherein the anti-tumor agent is docetaxel.

32. A method of treating abnormal cell growth in a mammal, the method comprising administering to the mammal a compound of formula 1: 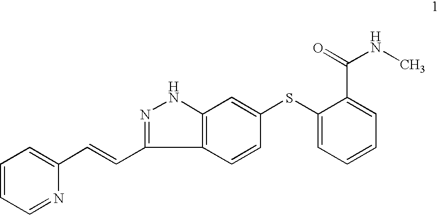
a pharmaceutically acceptable salt, solvate or prodrug thereof, or a mixture thereof, in an amount of no more than 30 mg per dose.

33. The method of claim 32, wherein the amount is from 0.5 to 30 mg.

34. The method of claim 32, wherein the amount is from 1 to 20 mg.

35. The method of claim 32, wherein the amount is from 1.5 to 15 mg.

36. The method of claim 32, wherein the amount is from 2 to 10 mg.

37. The method of claim 32, wherein the amount is from 2.5 to 8 mg.

38. The method of claim 32, wherein the amount is from 3 to 7 mg.

39. The method of claim 32, wherein the compound is administered orally.

40. The method of claim 32, wherein the compound is administered at a dosage frequency of at least once per day.

41. The method of claim 32, wherein the compound is administered at a dosage frequency of at least twice per day.

42. The method of claim 32, wherein the mammal fasts for at least two hours prior to the step of administering.

43. The method of claim 32, wherein the mammal fasts for at least two hours after the step of administering.

44. The method of claim 32, wherein the mammal fasts for at least two hours prior to the step of administering and at least two after the step of administering.

45. The method of claim 32, wherein the abnormal cell growth is cancer.

46. The method of claim 45, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

47. The method of claim 32, wherein the method further comprises co-administering an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, anti-androgens and mixtures thereof.

48. The method of claim 47, wherein the anti-tumor agent is docetaxel.
